(19) United States
(12) Patent Application Publication
(10) Pub. No.: US 2005/0177085 A1
(43) Pub. Date: Aug. 11, 2005

(54) Diabetic toe separators

(76) Inventors: Allan L. Green, Tamarac, FL (US); Michael Anthony, Coral Springs, FL (US)

Correspondence Address: Mark D. Bowen;Stearns Weaver Miller Weissler,, Alhadeff & Sitterson, P.A., 200 East Broward Boulevard, Suite 1900, Fort Lauderdale, FL, 33301, US

(21) Appl. No.: 10/773,881

(22) Filed: Feb. 6, 2004

(51) Int. Cl. A61F 5/00

(52) U.S. Cl. 602/30

(57) ABSTRACT

Toe separators for use by a diabetic patient to maintain the toes separated, even when wearing shoes, to prevent the development of sores on the toes resulting from abrasion, and to promote healing are disclosed. In a preferred embodiment, there is provided a generally flat sole insert for protecting the user's heel, wherein the insert is further provided with a plurality of upwardly projecting toe dividers for keeping the toes separated. The dividers effectively separate the user's toes to prevent rubbing while allowing the user to walk wearing shoes. In various alternate embodiments, there are disclosed individual soft toe caps that may be individually placed over one or more individual toes to protect and separate the toes. In still another alternate embodiment, the individual toe separators may be fabricated as double-wall caps formed by inner and outer layers of a soft, FDA approved silicon material, and adapted to be filled with water or other suitable liquid or a gel. According to another aspect, the present invention provides a foot protector to be worn on the foot of a patient. The foot protector preferably incorporates one or more water-filled toe protectors for separating the toes as generally described above. Each of the embodiments may define a plurality of ventilation holes formed by welding of inner and outer portions. Any of the embodiments disclosed herein may further include medicated powders to reduce friction and aid in healing ulcerations. The filled embodiments may further be cooled or heated to provide therapeutic cooling or warmth to the foot and toes.

### CROSS REFERENCE TO RELATED APPLICATIONS

N/A

### STATEMENT REGARDING FEDERALLY SPONSORED RESEARCH OR DEVELOPMENT

N/A

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office patent file or records, but otherwise reserves all copyrights.

### BACKGROUND OF THE INVENTION

1. Field of the Invention

The present invention relates generally to a toe separator for use on the foot of a patient and more particularly, to a toe separator effectively separates the toes of a diabetic patient while wearing a shoe to prevent sores resulting from the toes rubbing together.

2. Description of the Background Art

Many diabetics suffer from sores and irritations on the feet. As a result of problems associated with diabetes, sores on the feet of a diabetic are slow to heal and often susceptible to secondary infection. A particular problem is the development of sores resulting from rubbing of the toes while wearing shoes.

While there exist a number of foot protectors on the market today that are used by these patients, the prior art fails to disclose an apparatus for separating the toes of a diabetic to prevent rubbing of the toes, particularly while wearing shoes. In addition, a number of foot protectors are bulky, very heavy and difficult to use or walk on, particularly while wearing shoes.

Accordingly, there exists a need for an improved toe separator, particularly for use by diabetics suffering from sores of the feet. Such a device is also needed for patients after having foot surgery and/or bedridden individuals having ulcerative conditions, such as pressure sores, on the skin and underlying tissues and bone of the feet and toes as a result of extended contact of the skin with bedding materials.

### BRIEF SUMMARY OF THE INVENTION

The present invention provides a toe separator for use by a diabetic patient to maintain the toes separated, even when wearing shoes, to prevent the development of sores on the toes resulting from abrasion, and to promote healing. In a preferred embodiment, there is provided a generally flat sole insert for protecting the user's heel, wherein the insert is further provided with a plurality of upwardly projecting toe dividers for keeping the toes separated. The dividers effectively separate the user's toes to prevent rubbing while allowing the user to walk wearing shoes. In an alternate embodiment, there is disclosed individual soft foam toe caps that may be individually placed over one or more individual toes to protect and separate the toes. In still another alternate embodiment, the individual toe separators may be fabricated as double-wall caps formed by inner and outer layers of a soft, FDA approved silicon material, and adapted to be filled with water or other suitable liquid or a gel. According to another aspect, the present invention provides a foot protector to be worn on the foot of a patient. The foot protector preferably incorporates one or more water-filled toe protectors for separating the toes as generally described above. Each of the embodiments may define a plurality of ventilation holes formed by welding of inner and outer portions. Any of the embodiments disclosed herein may further include medicated powders to reduce friction and aid in healing ulcerations. The filled embodiments may further be cooled or heated to provide therapeutic cooling or warmth to the foot and toes.

Accordingly, it is an object of the present invention to provide an improved toe separator particularly suitable for use by diabetics suffering from sores, bunions, or other abnormalities of the feet.

Still another object of the present invention is to provide a toe separator fabricated from a soft silicone material.

Yet another object of the present invention is to provide an apparatus for separating the user's toes that is capable of being worn with shoes.

In accordance with these and other objects, which will become apparent hereinafter, the instant invention will now be described with particular reference to the accompanying drawings.

### BRIEF DESCRIPTION OF THE SEVERAL VIEWS OF THE DRAWINGS

The drawings furnished herewith illustrate a preferred construction of the present invention in which the above advantages and features are clearly disclosed as well as other which will be readily understood from the following description of the illustrated embodiment.

FIG. 1 is a partial perspective view of a human foot;

FIG. 2 depicts toe caps in accordance with an alternate embodiment of the present invention;

FIG. 3 is a bottom view thereof;

FIG. 4 is a perspective view of a toe separator;

FIG. 5 is a perspective view of an alternate embodiment toe separator;

FIG. 6 is a sectional view thereof;

FIG. 7 is a perspective view of an alternate embodiment toe separator;

FIG. 8 is a partial sectional view thereof;

FIG. 9 depicts a preferred embodiment toe separator positioned on a human foot;

FIG. 10 is a bottom perspective view thereof;

FIG. 11 is an alternate embodiment thereof having shields for protecting the top surface of the foot;

FIG. 12 is a perspective view of the toe separator;

FIG. 13 is a perspective view of the alternate embodiment toe separator;

FIG. 14 is another alternate embodiment;

FIG. 15 is still another alternate embodiment;

FIGS. 16 and 17 are sock-type alternate embodiments; and

FIG. 18 is another alternate embodiment.

### DETAILED DESCRIPTION OF THE INVENTION

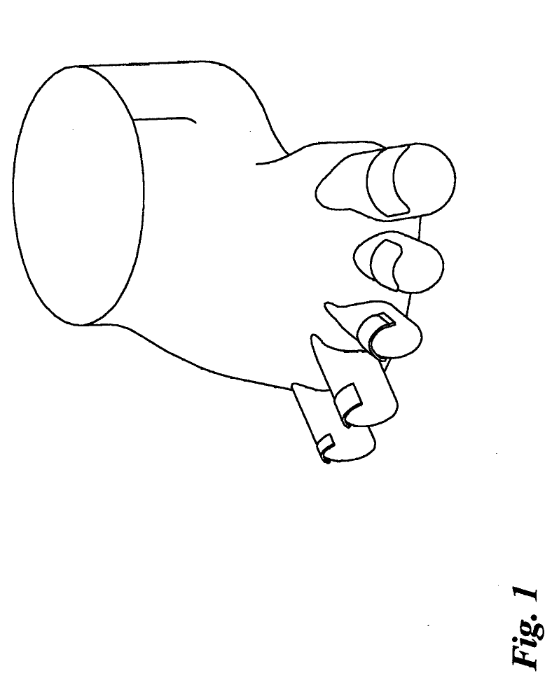

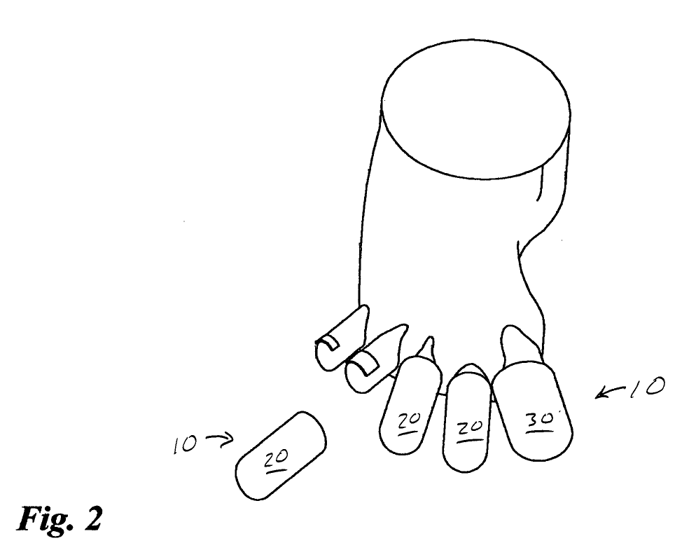

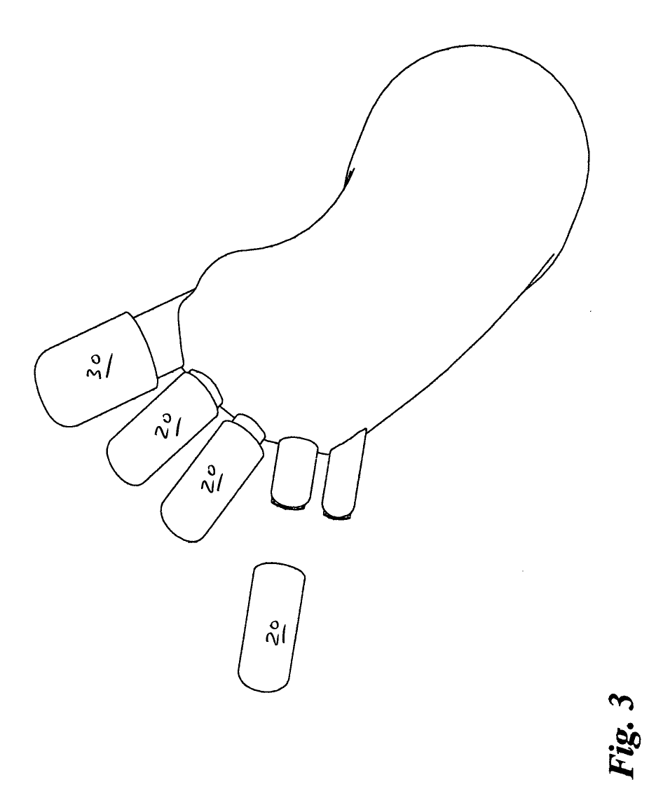

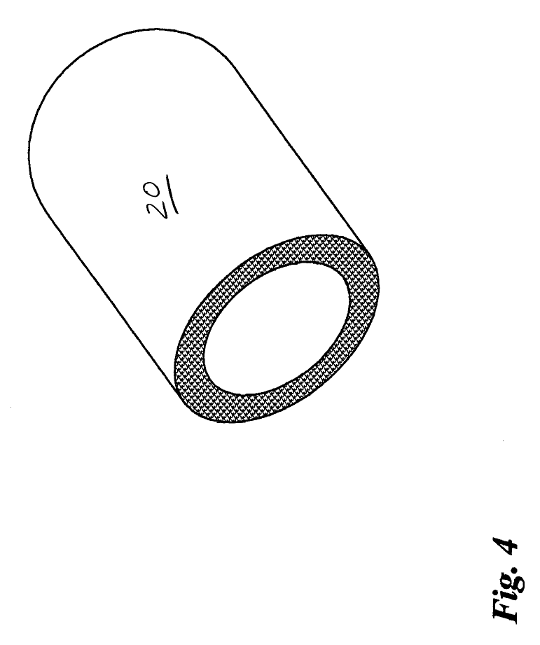

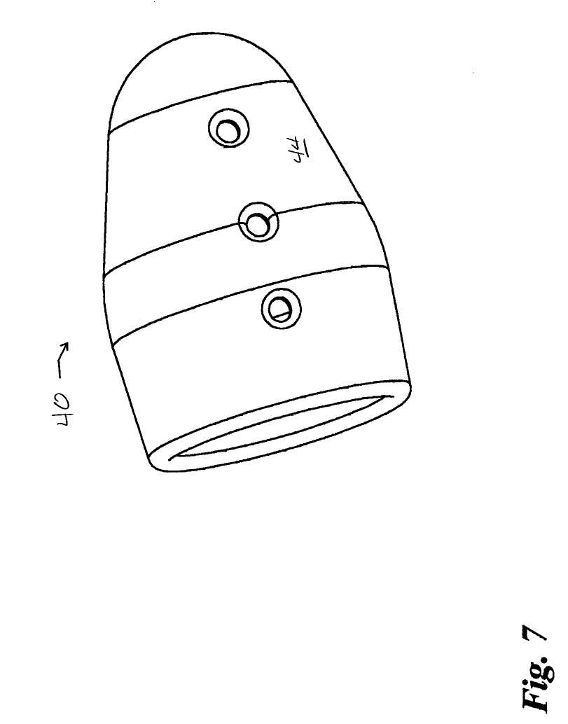

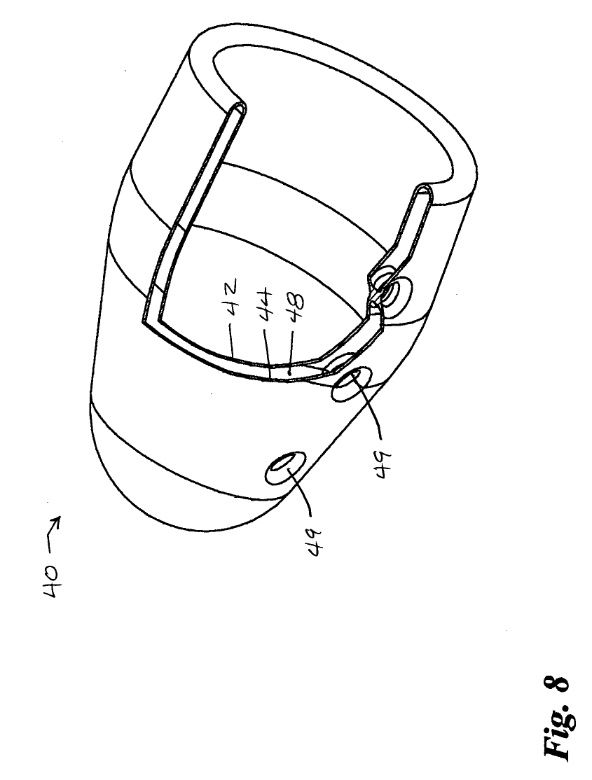

With reference to the drawings, there is depicted a preferred and various alternate embodiment toe separators according to the present invention. Turning initially to FIGS. 1 through 8, there are depicted several embodiments of toe separators in the form of toe caps, generally referenced as 10. FIGS. 2 and 3 depict toe caps 10 adapted to be worn on one or more toes so as to substantially cover the distal end of the toes thereby maintaining separation thereof. As further depicted in FIGS. 4 through 8, each toe cap 10 may be formed as a generally cylindrical body having an outer wall 12 and an inner wall 14 defining a toe receiving recessed volume 16. Each toe cap 10 is preferably sized to be comfortably worn on one of the toes of a user's foot.

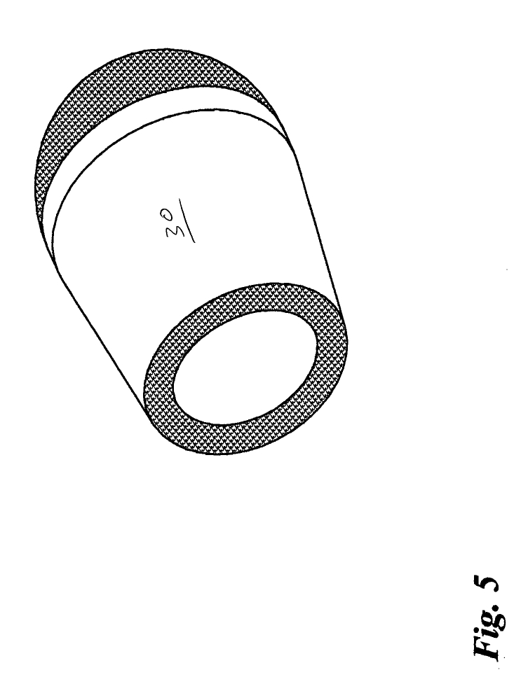

As should be apparent, each toe cap 10 comprises a body sized to substantially cover at least the distal end of the toe of the user. Each toe cap 10 may be fabricated either from a foam or sponge-like material. In addition, each toe cap 10 may be fabricated from a soft FDA approved material, such as silicone, in a double wall configuration wherein an interstitial space formed between inner and outer walls is capable of being filled with water, other suitable liquid, or gel. FIGS. 4 and 5 depict alternate toe cap structures, referenced as 20 and 30 respectively, formed of a foam material. As seen in FIG. 4 toe cap 20 is suitably shaped to snugly fit the second through fifth (“little”) toes. As seen in FIG. 5, toe cap 30 is suitably shaped to snugly fit the generally larger first (“big”) toe.

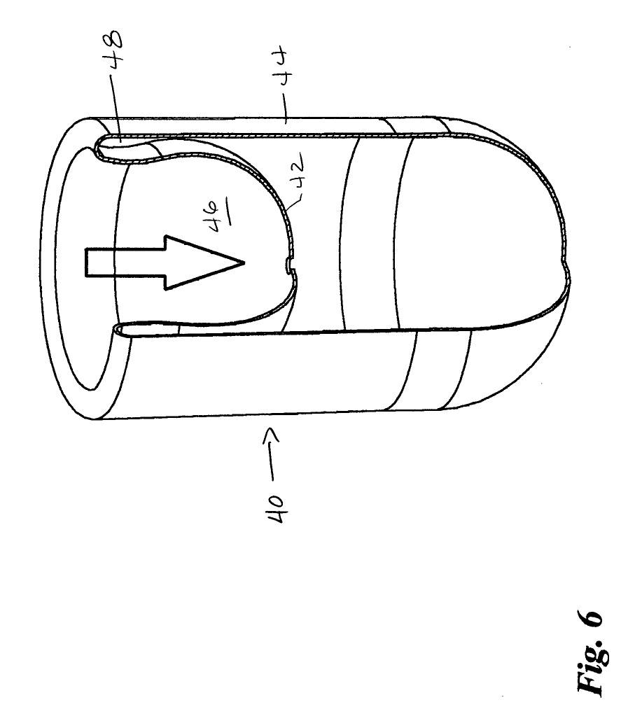

FIGS. 6 through 8 depict an alternate embodiment double wall toe cap, generally referenced as 40, having inner and outer sleeve members, referenced as 42 and 44 respectively. Inner and outer sleeve members 42 and 44 are sealingly connected to form a double wall tubular toe protector defining an interior volume 46 and an interstitial space 48 disposed between inner and outer sleeve members 42 and 44. As should be apparent, toe cap 40 is preferably sized and shaped to conform to the size and shape of a human toe. According to significant aspect of the present invention, toe cap 40, and particularly the interstitial space 48 defined between inner and outer sleeve members 42 and 44, is preferably filled with a liquid, such as water, a gel substance, or any other suitable filling agent. Toe cap 40 further includes air vents 49 formed by welding inner and outer sleeve members to form apertures. When filled, toe cap 40 is inserted over one of the user's toes and may be held in place by particular sizing for a snug fit. Accordingly, use of one or more toe caps effectively protect the toes from abrasive rubbing together by maintaining separation.

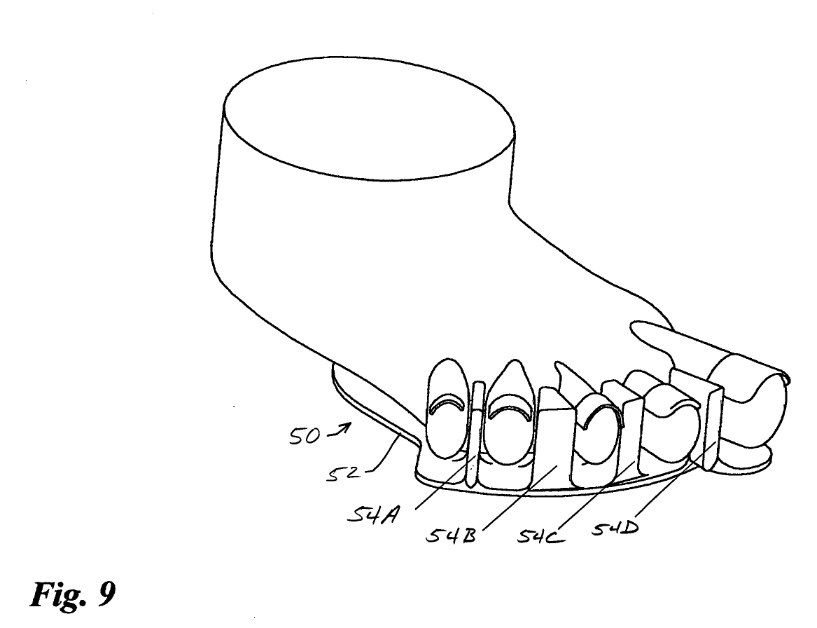

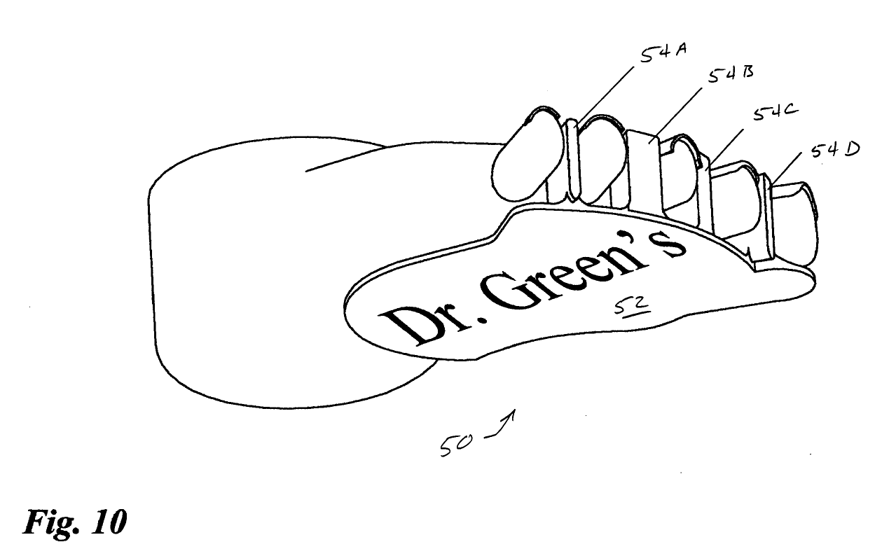

FIGS. 9-13 depict alternate embodiments wherein the improvements disclosed herein above relating to toe separation are incorporated into a shoe insert, generally referenced as 50. Insert 50 is preferably fabricated using similar medically approved materials, such as silicone or a suitable foam. In another embodiment, insert 50 may be fabricated as a water-filled double walled structure. As best depicted in FIGS. 9 and 10, insert 50 comprises a sole insert 52 and a plurality of toe dividers 54A-54D projecting upwardly from sole insert 52. Insert 50 is preferably sized and shaped in the form of an insert capable of being received within a conventional shoe. Toe dividers 54 are sized and positioned on sole insert 52 so as to provide soft and/or cushioned barriers between the user's toes.

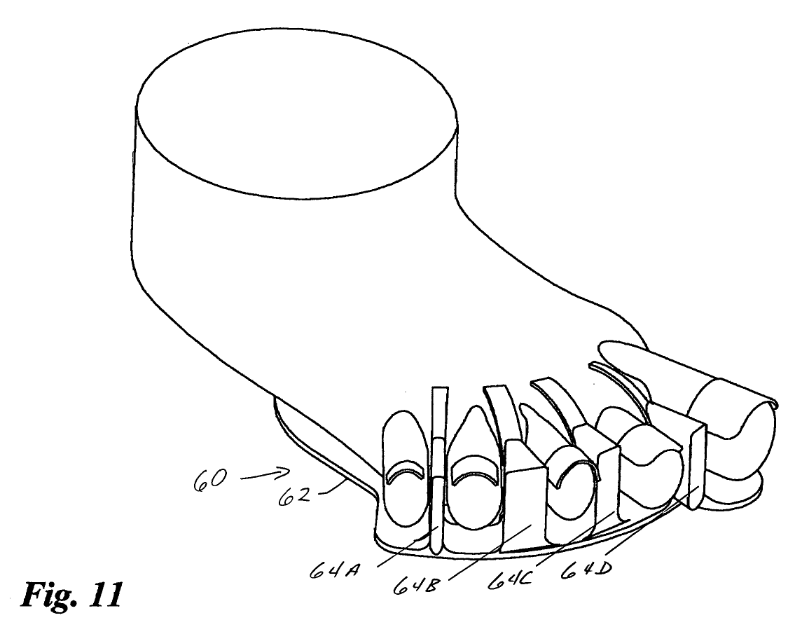

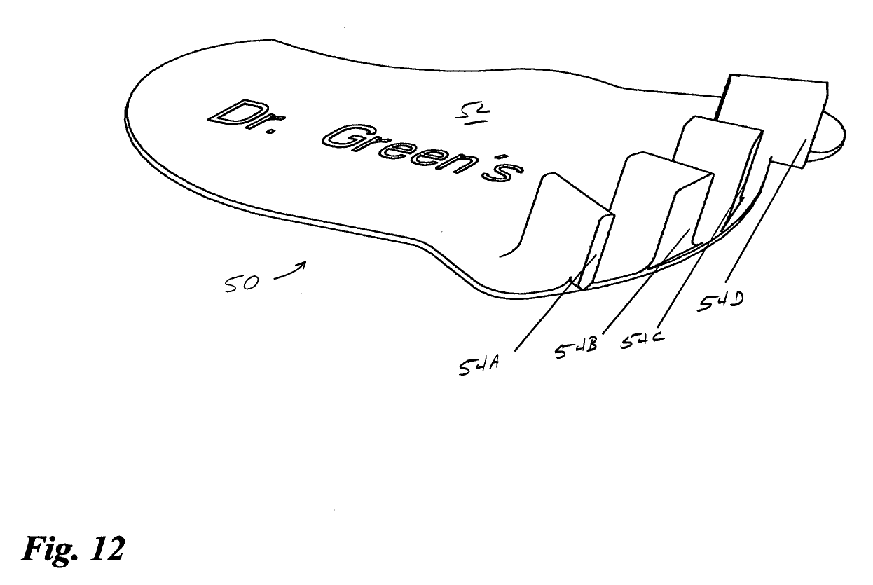

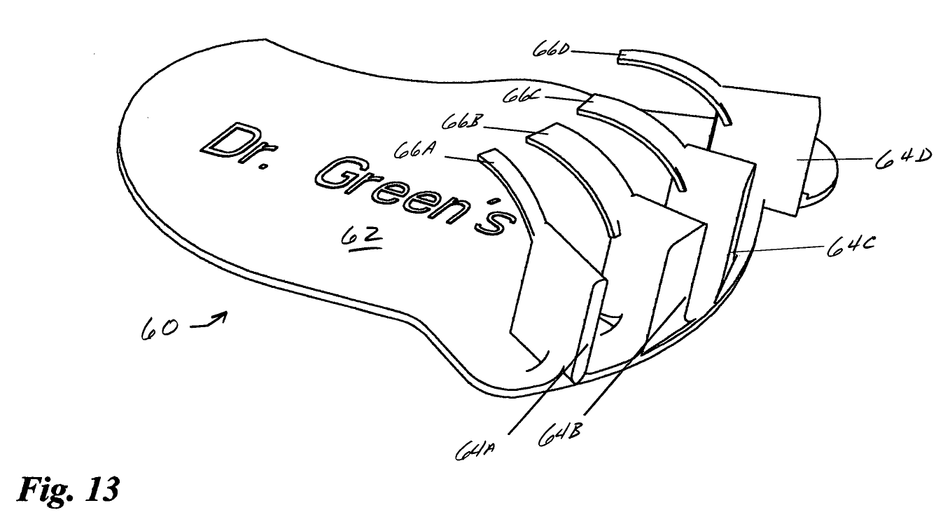

FIGS. 11 and 12 depict an alternate embodiment insert, referenced as 60. Insert 60 comprises a sole insert 62 and a plurality of toe dividers 64A-64D projecting upwardly from sole insert 64. In similar fashion, insert 60 is preferably sized and shaped in the form of an insert capable of being received within a conventional shoe. Toe dividers 64 are sized and positioned on sole insert 62 so as to provide soft and/or cushioned barriers between the user's toes. In addition, sole insert 62, and more particularly toe dividers 64 further include rearwardly projecting tabs 66A-66D for helping to maintain sole insert 62 securely attached to the user's foot while further protecting the top of the foot from abrasion. FIG. 13 depicts sole insert 60 without the user's foot inserted thereon.

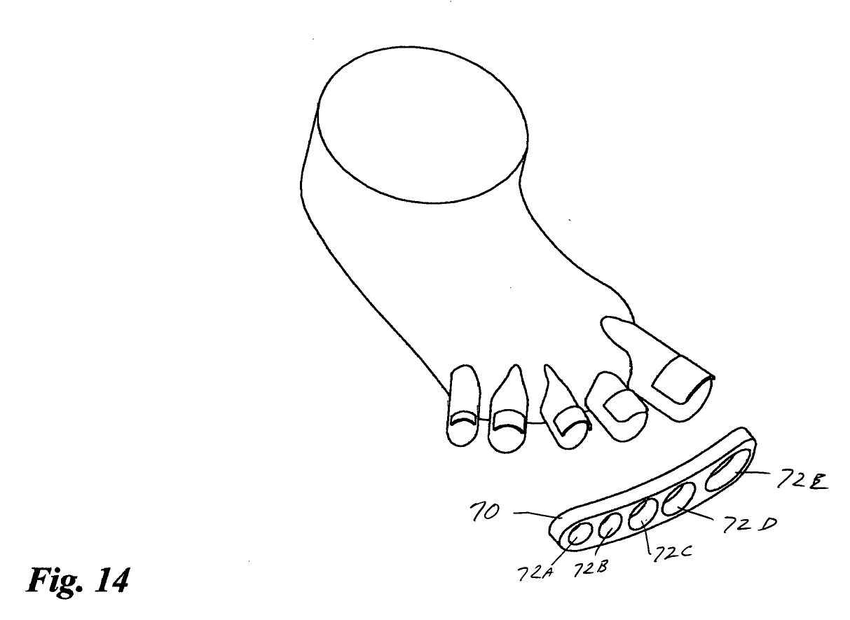

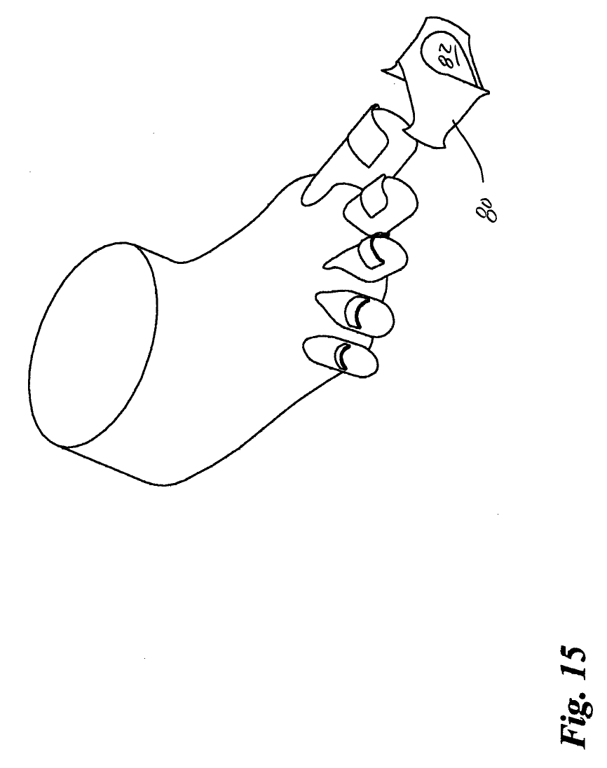

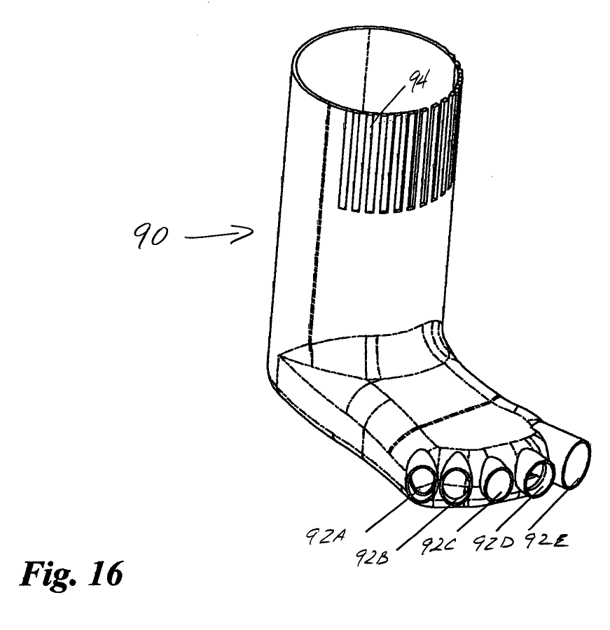

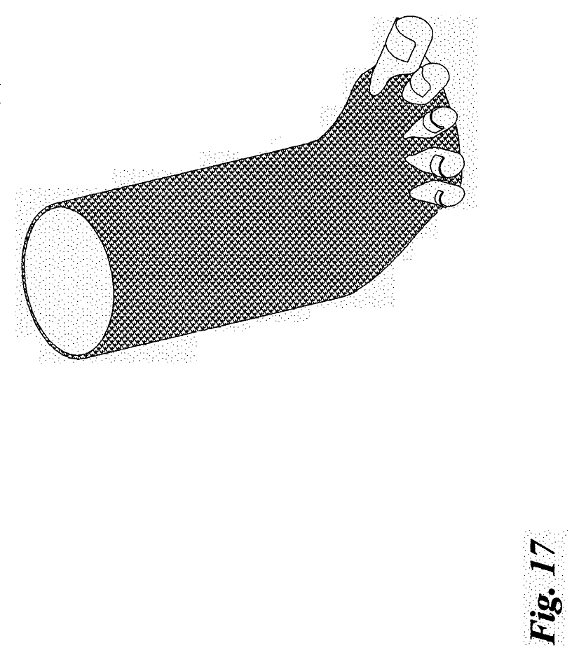

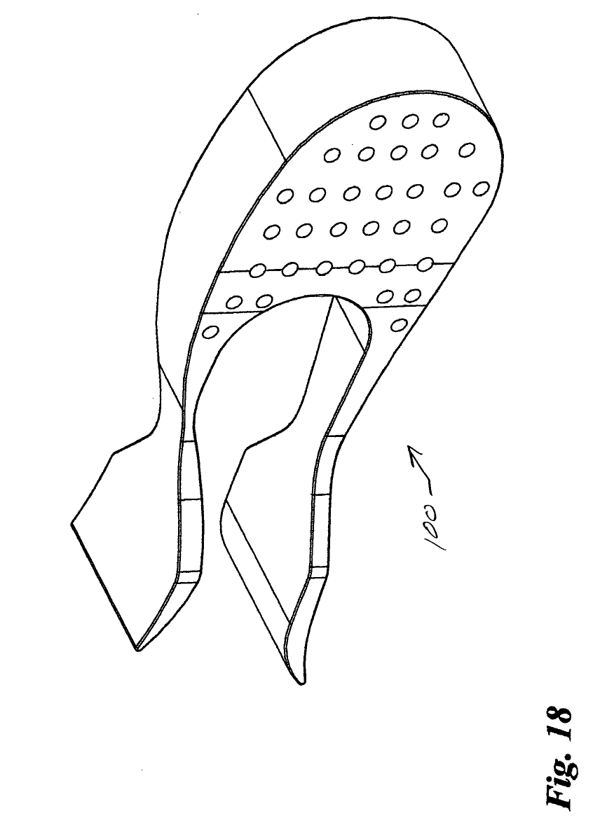

FIG. 14 depicts another alternate embodiment toe separator 70 comprising an elongate apparatus defining five (5) toe-receiving apertures, referenced as 72A-72E therein. As should be apparent, each aperture 72 is sized and positioned for snugly receiving a toe of the user's foot. FIG. 15 depicts yet another alternate embodiment toe separator 80 comprising a slip on device defining a generally tubular toe-receiving opening 82. FIG. 16 depicts yet another embodiment toe separator 90 in the form of a sock having individual toe compartments, referenced as 92A-92E. Various embodiments are contemplated wherein either the entire device, any one toe, combination of toes, or all toes may be adapted with the water-filled cushioned structure and/or be fabricated from foam. In addition, the embodiment depicted in FIG. 16 discloses air vents 94 formed in the structure for ventilation and comfort. Air vents 94 are preferably formed by welding the inner and outer layers into any suitable sized or shaped ventilation openings. FIG. 17 depicts a foam embodiment of the device depicted in FIG. 16. FIG. 18 depicts a toe separator 100 particularly adapted for protecting the upper and lower portions of the toe and the tip of the toe.

The instant invention has been shown and described herein in what is considered to be the most practical and preferred embodiment. It is recognized, however, that departures may be made therefrom within the scope of the invention and that obvious structural and/or functional modifications will occur to a person skilled in the art.

1. A toe separator to be worn on the foot of a patient, the toe separator comprising:
a sole plate sized and shaped to conform to the sole of a human foot;
said sole plate including a front portion including four upwardly projecting toe dividers.

2. A toe separator according to claim 1, wherein said sole plate includes a foam upper surface.

3. A toe separator according to claim 1, wherein said toe dividers each include a foam outer surface.

4. A toe separator according to claim 1, wherein said toe dividers each include medicated powder.

5. A toe separator according to claim 1, wherein sole plate comprises a double wall structure having upper and lower walls in spaced relation defining an interstitial space.

6. A toe separator according to claim 5, further including a substance contained within said interstitial space.

7. A toe separator according to claim 6, wherein said substance is a gel.

8. A toe separator according to claim 6, wherein said substance is liquid.

9. A toe separator according to claim 8, wherein said liquid is water.

10. A toe separator to be worn on the foot of a patient, the toe separator comprising:
a sole plate insert sized and shaped to conform to the bottom of the patient's foot;
said sole plate having a front edge including four upwardly projecting toe dividers; and
said toe dividers each including an upper surface and rearwardly projecting tab connected to said upper surface.

11. A toe separator according to claim 10, wherein said sole plate includes a foam upper surface.

12. A toe separator according to claim 10, wherein said toe dividers each include a foam outer surface.

13. A toe separator according to claim 10, wherein said toe dividers each include medicated powder.

14. A toe separator according to claim 10, wherein sole plate comprises a double wall structure having upper and lower walls in spaced relation defining an interstitial space.

15. A toe separator according to claim 14, further including a substance contained within said interstitial space.

16. A toe separator according to claim 15, wherein said substance is a gel.

17. A toe separator according to claim 16, wherein said substance is liquid.

18. A toe separator according to claim 17, wherein said liquid is water.
